(19) United States
(12) Patent Application Publication
(10) Pub. No.: US 2009/0005831 A1
(43) Pub. Date: Jan. 1, 2009

(54) METHOD, APPARATUS AND PROTOCOL FOR SCREENING APPROPRIATE PATIENT CANDIDATES AND FOR CARDIAC RESYCHRONIZATION THERAPY (CRT), DETERMINING CARDIAC FUNCTIONAL RESPONSE TO ADJUSTMENTS OF VENTRICULAR PACING DEVICES AND FOLLOW-UP OF CRT PATIENT OUTCOMES

(76) Inventors: Lon P. Wilson, Irwin, PA (US); Herbert D. Clauhs, Sellersville, PA (US); Floyd M. Casaday, Indiana, PA (US); Keith E. Loiselle, Gibsonia, PA (US)

Correspondence Address: MCGUIREWOODS, LLP, 1750 TYSONS BLVD, SUITE 1800, MCLEAN, VA, 22102, US

(21) Appl. No.: 12/130,932

(22) Filed: May 30, 2008

Related U.S. Application Data
(60) Provisional application No. 60/941,522, filed on Jun. 1, 2007.
(60) Provisional application No. 60/973,687, filed on Sep. 19, 2007.

(51) Int. Cl. A61N 1/00

(52) U.S. Cl. 607/27

(57) ABSTRACT

An apparatus, a method and a protocol for optimizing an implanted device in a candidate. The apparatus comprises a first sensor configured to sense a tracing signal, a transducer configured to capture an image of a region of interest, where the image is captured in synchronism with the tracing signal, and a determiner configured to determine a cardiac functional value based on the image and the tracing signal. A parameter of the implanted device is adjusted based on the cardiac functional value.

### CROSS REFERENCE TO PRIOR APPLICATIONS

This application claims priority and the benefit thereof from U.S. Provisional Application No. 60/941,522, filed on Jun. 1, 2007, and U.S. Provisional Application No. 60/973,687, filed Sep. 19, 2007, which are hereby incorporated by reference for all purposes as if fully set forth herein.

### BACKGROUND

1. Field

This disclosure relates to a method, a protocol and an apparatus for noninvasive screening and evaluating cardiac or cardiovascular health. In particular, the present disclosure relates to measurement and evaluation of cardiac function as it relates to a sequence of contraction and interconnected conduction through parametric imaging. Still more particularly, the disclosure relates to measurement and evaluation of changes in cardiac function brought about by changes in ventricular pacing devices.

2. Related Art

Sudden cardiac death (SCD) is responsible for hundreds of thousands of deaths annually in the United States. In many of these deaths, the persons were asymptomatic. Clinical indicators for patients at risk for SCD include, for example, post myocardial infarct, congestive heart failure, documented sustained or non-sustained ventricular tachycardia, family history of SCD, family history of coronary heart disease, coronary heart disease, shortness of breath, syncope, cardiomyopathy, coronary heart disease risk factors, and the like. Since many patients that are at risk for SCD may be asymptomatic, it is important to screen, identify and evaluate those individuals for appropriate candidates for preventive measures, such as, for example, implantation of an internal cardiac defibrillator (ICD), ventricular or bi-ventricular (Bi-V) cardiac device.

Further, Cardiac Resynchronization Therapy (CRT) is a recent treatment option of medically refractory Heart Failure (HF) by Biventricular Pacing in selected patients. Pacemaker treatment for severe HF started at the beginning of the 1980's, however a new era of CRT by biventricular pacing of both right and left ventricles has rapidly developed in recent years. There are a large number of HF patients suffering from intra- and interventricular asynchronous contraction and relaxation assumed by the surface ECG further deteriorating an already hemodynamically compromised left ventricle. Biventricular pacing is assumed to provide a more coordinated pattern of ventricular contraction and reduce intraventricular and interventricular asynchrony. Long-term clinical benefits by CRT has been proven in several studies. Even though CRT has proven to improve several homodynamic and clinical indices in almost 70% of the patients, it is still difficult to define responders to CRT treatment, and most commonly composite clinical endpoints have been used. Of major interest is to decrease the number of clinical non responders, which is described to approximately 30%.

Cardiac Resynchronization Therapy (CRT) Optimization occurs to ensure adequate device function in a CRT patient with persistent or worsening symptoms. This leads to evaluation of AV (Atrioventricular) and VV (Interventricular) delay. Restoration of optimal AV timing may improve systolic performance by optimizing Left Ventricular preload. Also of great interest is to find an accurate and reproducible method to optimize these ventricular devices to increase the number of responders and further improve the efficiency and benefit of these devices in those who do respond.

### SUMMARY

In one aspect of the invention, an apparatus for optimizing an implanted device in a candidate is provided. The apparatus comprises a first sensor configured to sense a tracing signal; a transducer configured to capture an image of a region of interest, where the image is captured in synchronism with the tracing signal; and a determiner configured to determine a cardiac functional value based on the image and the tracing signal, wherein a parameter of the implanted device is adjusted based on the cardiac functional value. The apparatus may comprise a second transducer configured to capture a second image of the region of interest, where the second image is captured in synchronism with the tracing signal; a third transducer configured to capture a third image of the region of interest, where the third image is captured in synchronism with the tracing signal; and/or a support member configured to moveably support the transducer and the second transducer. The transducer and the second transducer may be moveable based on the region of interest.

The apparatus may further comprise a mobile candidate support; and a mobile transducer array support, wherein the mobile candidate support and the mobile transducer array support are lockably engageable. The mobile candidate support platform may comprise an adjustable candidate support member. The adjustable candidate support member may be configured in a seat position, a supine position, a recumbent position, or a vertical position.

The apparatus may further comprise a physical resistance device configured to provide a resistive force. The physical resistance device may comprise at least one foot pedal.

The apparatus may further comprise a broad-range post configured to provide substantially precise movement of the support member, or a narrow-range post configured to provide substantially precise movement of the support member. The narrow-range post may comprise a hinged bracket. The broad-range post may comprise a motorized post. The mobile candidate support and the mobile transducer array support may be lockable by at least one locking pin. The tracing signal may comprise an electrocardiogram tracing update signal.

According to a further aspect of the disclosure, a method for evaluating an implanted device in a candidate is provided. The method comprises sensing a tracing signal; capturing an image of a region of interest in synchronism with the tracing signal; determining a cardiac functional value based on the tracing signal and the image; and adjusting a parameter of the implanted device based on the cardiac functional value. The method may further comprise capturing a second image of the region of interest in synchronism with the tracing signal; capturing a third image of the region of interest in synchronism with the tracing signal; moveably supporting a first transducer and a second transducer to capture said image and said second image; moving the first transducer and the second transducer based on the region of interest; moving a candidate support platform to position a candidate proximate the first transducer and the second transducer; and/or providing a resistive force to a candidate to cause an elevated heart rate. The moveably supporting may comprise providing substantially precise large scale movement of a support member; and providing substantially precise small scale movement of the support member. The small scale movement may comprise moving a hinged bracket. The large scale movement may comprise controlling a motorized post. The method may further comprise determining whether cardiac synchronization is improved based on the cardiac functional value.

According to a still further aspect of the disclosure, a computer readable medium comprising a program for evaluating an implanted device in a candidate is provided. The medium comprises a sensing code segment that, when executed by a computer, causes sensing a tracing signal; an image capturing code segment that, when executed by the computer, causes capturing an image of a region of interest in synchronism with the tracing signal; a cardiac functional value determining code segment that, when executed by the computer, causes determining a cardiac functional value based on the tracing signal and the image; a parameter adjusting code segment that, when executed by the computer, causes adjusting a parameter of the implanted device based on the cardiac functional value; and an improvement determining code segment that, when executed by the computer, causes determining whether a cardiac synchronization improvement is greater than or equal to a baseline based on the cardiac functional value.

Additional features, advantages, and embodiments of the disclosure may be set forth or apparent from consideration of the following detailed description, drawings, and claims. Moreover, it is to be understood that both the foregoing summary of the disclosure and the following detailed description are examples and are intended to provide further explanation without limiting the scope of the disclosure as claimed.

### BRIEF DESCRIPTION OF THE DRAWINGS

The accompanying drawings, which are included to provide a further understanding of the disclosure, are incorporated in and constitute a part of this specification, illustrate embodiments of the disclosure and together with the detailed description serve to explain the principles of the disclosure. No attempt is made to show structural details of the disclosure in more detail than may be necessary for a fundamental understanding of the disclosure and the various ways in which it may be practiced. In the drawings:

FIG. 1 shows an example of a typical electrocardiogram tracing of an electrical heart function signal that may be generated and spread through a heart;

FIG. 2 shows an example of a candidate screening/optimization apparatus (CSA) according to the disclosure;

FIG. 3 shows an example of a process for screening candidates for implantable CSDs, according to the disclosure;

FIG. 4 shows another example of a candidate screening/optimization apparatus (CSA) according to the disclosure;

FIG. 5 shows an example of an candidate screening/optimization system (CSS) for exposing a candidate to an increased stimulant, such as, for example, low level exercise, according to an embodiment of the disclosure;

FIG. 6 shows an example of a candidate platform according to a preferred embodiment of the disclosure;

FIG. 7 shows a further example of a top view of an aspect of the preferred embodiment of the disclosure;

FIG. 8 shows an example of a process for evaluating heart function in candidates with implanted CSDs and for optimizing the implanted CSDs, according to an embodiment of the disclosure;

FIG. 9 shows another example of a candidate screening/optimization system (CSS) according to a further embodiment of the disclosure; and

FIG. 10 shows a rear view of the candidate screening/optimization system (CSS) of FIG. 9.

### DETAILED DESCRIPTION

The embodiments of the disclosure and the various features and details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure teaching principles of the disclosed embodiments. The examples used herein are intended merely to facilitate an understanding of ways in which the disclosure may be practiced and to further enable those of skill in the art to practice disclosed the embodiments. Accordingly, the examples and embodiments herein should not be construed as limiting. Moreover, it is noted that like reference numerals represent similar parts throughout the several views of the drawings.

ICDs are small devices that are typically implanted in patients at a location below the collarbone. An ICD continuously monitors the rhythm of a patient's heart and applies a jolting electrical signal to the heart when the heart is determined to beat too quickly, thereby restoring operation of the heart to a normal rhythm. When the heart is determined to beat to slowly, the ICD may function as a pace maker.

Cardiac resynchronization therapy (CRT) is a recent treatment option of medically refractory heart failure (HF) by biventricular pacing in selected patients. Pacemaker treatment for severe HF started at the beginning of the 1980's, however a new era of CRT by biventricular pacing of both right and left ventricles has rapidly developed in recent years. There are a large number of HF patients suffering from intraventricular and interventricular asynchronous contraction and relaxation assumed by a surface electrocardiogram (ECG) further deteriorating an already hemodynamically compromised left ventricle. Biventricular pacing is assumed to provide a more coordinated pattern of ventricular contraction and reduce intraventricular and interventricular asynchrony. Long-term clinical benefits by CRT have been proven in several studies. Even though CRT has proven to improve several hemodynamic and clinical indices in almost seventy percent (70%) of the patients, it is still difficult to define responders to CRT treatment.

An apparatus is provided, according to an embodiment of the disclosure, for screening candidates that are at risk for SCD, or for evaluating and optimizing an implanted cardiac stimulation device (CSD), such as, for example, but not limited to, a CRT device or an ICD device. The apparatus may be used, for example, but not limited to, determining and selecting candidates with mechanical LV asynchrony prior to implanting the cardiac stimulation device (CSD). The apparatus may also be used, for example, for evaluating and optimizing the cardiac stimulation device (CSD) after it has been implanted in the candidate.

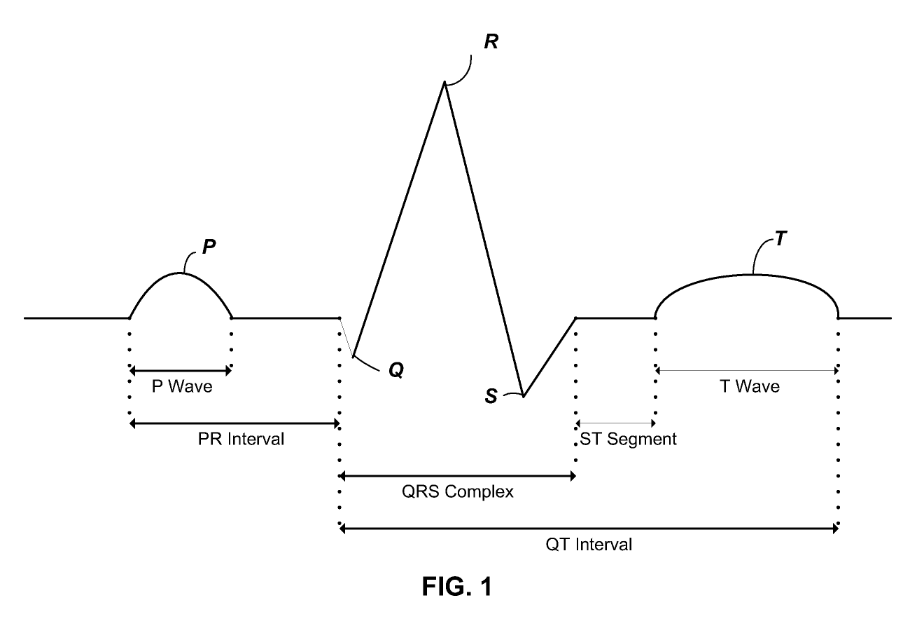

FIG. 1 shows an example of a typical electrocardiogram tracing of an electrical heart function signal that may be generated and spread through a heart. The electrical heart function signal generated by the heart may include, as depicted in FIG. 1, a P wave, a Q wave, an R wave, an S wave and a T wave. Generally, the current is generated by the sinoatrial node (SA node) of the heart and propagated to the myocardium, which contracts after stimulation. Orderly stimulation of the myocardium provides for efficient contraction of the heart. Under normal conditions, the generated current is propagated throughout the right atrium and through Bachman's Bundle to the left atrium, stimulating the myocardium of the atria to contract. The signal conducted throughout the atria takes on the form of the P wave shown in FIG. 1. The signal spreads throughout the atria, spreading from the SA node to the AV node via specialized pathways known as internodal tracts. The AV node delays the signal for the PR Interval, before splitting the signal into two branches and propagating the signal to the left and right ventricles of the heart. The spread of the signal through the ventricular myocardium produces the QRS complex waves. Repolarization of the ventricles is manifested in the T wave.

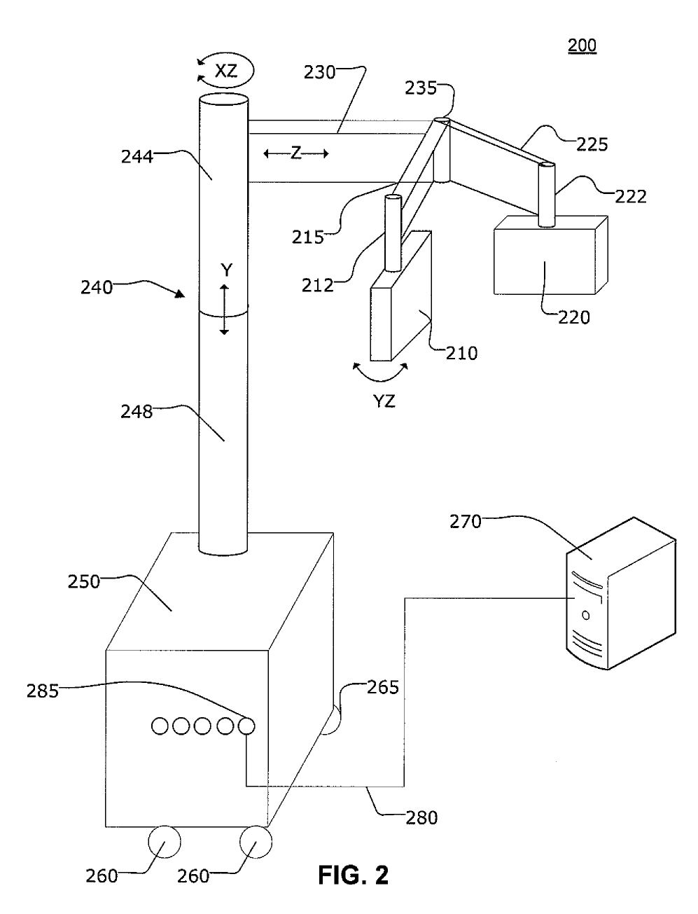

FIG. 2 shows an example of a candidate screening apparatus (CSA) 200 according to an embodiment of the disclosure. The CSA 200 includes a pair of transducers 210, 220, a pair of transducer support members 215, 225, an assembly support member 230, a dynamic support column 240, a base 250 and a computer 270, which may be connected to the base 250 via a connection channel 280 and a connector 285.

The dynamic support column 240 may include an upper column portion 244 and a lower column portion 248, where each or both of the column portions 244, 248 may be manually or automatically moveable in a vertical up/down direction Y (e.g., perpendicular to a floor surface) through a manual drive mechanism (not shown), a motorized drive mechanism (not shown), or a hydraulic mechanism (not shown). Moreover, the column portions 244, 248 may be manually or automatically rotatable in an X-Z plane, i.e., in a plane parallel to a floor surface (not shown). The upper column portion 244 may be connected to the assembly support member 230 and be configured to facilitate manual or automatic movement of the assembly support member 230 in a horizontal Z direction.

The assembly support member 230 may be coupled to the transducer support members 215, 225 through a pivotal member 235. The pivotal member 235 may provide for manual or automatic movement of the transducer support members 215, 225 in the X-Z plane. Moreover, the transducer support members 215, 225 may be individually or jointly moveable in the X-Z plane. The transducer support members 215, 225 may be coupled to transducers 210, 220, through a pair of pivotal members 212, 222, respectively. The pivotal members 212, 222 may provide for manual or automatic rotationally movement of the transducers 210, 220, in the X-Z plane and/or manual or automatic pivotal movement of the transducers 210, 220, in the Y-Z plane.

The base 250 may be affixed to, for example, a pair of front coaster members 260, and a pair of rear coaster members 265 (only one of which is shown in FIG. 2), enabling movement of the CSA 200 in any direction parallel to a floor surface (not shown). The coaster members 260, 265 may include wheels that are moveable in a plane parallel to the floor in three-hundred-sixty (360) degrees. The coaster members 260, 265 may be driven by an internal driver, such as, e.g., an electric motor, or as a result of an external force applied to the CAS 200.

The computer 270 may include any machine, device, circuit, component, or module, or any system of machines, devices, circuits, components, modules, or the like, which are capable of manipulating data according to one or more instructions, such as, for example, without limitation, a processor, a microprocessor, a central processing unit, a general purpose computer, a personal computer, a laptop computer, a palmtop computer, a notebook computer, a desktop computer, a workstation computer, a server, or the like, or an array of processors, microprocessors, central processing units, general purpose computers, personal computers, laptop computers, palmtop computers, notebook computers, desktop computers, workstation computers, servers, or the like.

It is noted that the each of the transducer support members 215, 225, the assembly support member 230, the dynamic support column portions 244, 248 and the base 250 may be configured from single, unitary members, or from a plurality of telescopic members that are configured to extend or retract, without departing from the scope or spirit of the disclosure. Moreover each of the support members 215, 225, the assembly support member 230, the dynamic support column portions 244, 248, the base 250 and/or the coasters 260, 265 may be locally or remotely controlled.

Each of the transducers 210, 220 may include, for example, but are not limited to, an analog or digital gamma camera that is capable of sensing and capturing radiation signals emitted by a radionuclide tracer, which may have been intravenously injected into a patient candidate. For example, each of the transducers 210, 220 may include, but is not limited to, an array of compact position sensitive photomultiplier tubes (PMTs) (not shown), a scintillation array for breaking down light into discrete light segments (not shown), a light guide array (not shown) or element (not shown) for directing the light segments to the PMT array, a collimator (not shown) and a dedicated processor (not shown) for processing image data that is captured by the transducers 210, 220. The PMTS and/or the scintillation array may be arranged in n×m arrays, where n and m are nonzero integers and where n may equal m.

Further, each of the transducers 210, 220 may be configured to provide self-correcting positional alignment with respect to a particular area in the candidate and with respect to other transducers. The transducers 210, 220 may be further configured to provide synchronic-simultaneous imaging per transducer.

ECG tracing updates with corresponding Blood Pool Imaging output data (Global and Regional) may be provided from the base 250 to the computer 270 through the communication medium 280, which may include a wireless, a wired, or a combination of wireless-wired communication medium. The ECG tracing updates with corresponding Blood Pool output data may be displayed in a multi-screen format on a display (not shown) of, for example, the computer 270, for wall motion and phasic analysis-comparative updates per sequence of optimization, mechanical LV Asychrony and screening of candidates for SCD.

Further, additional transducers may be provided in the CAS 200, which may include gamma cameras. For example, the CAS 200 may include three, four, five, six, or more transducers that are manually or automatically moveable, without departing from the scope or spirit of the disclosure.

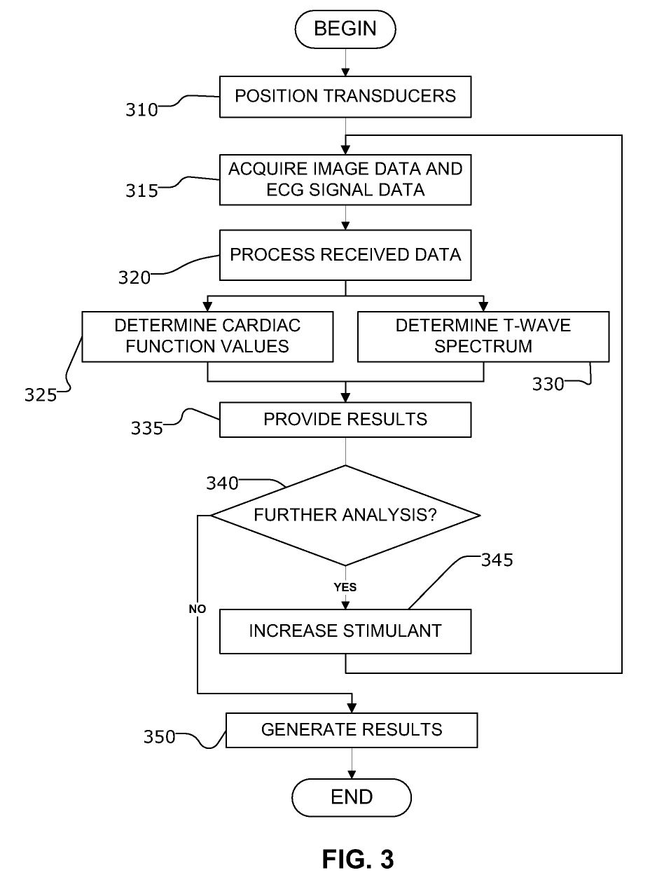

FIG. 3 shows an example of a process for screening candidates for implantable CSDs, according to an embodiment of the disclosure.

Referring to FIG. 3, after a candidate has been injected with one of several radionuclide tracers, one or more transducers may be positioned proximate the candidate and a plurality of sensors (such as, e.g., electrodes) may be connected to the candidate (Step 310). The sensors, which may include high-resolution electrodes, may be coupled to an electrocardiogram (ECG). The transducers may be positioned in close proximity to the chest and heart of the candidate from several different projections, thereby making it possible to acquire data for a standard multi-gated acquisition (MUGA) analysis and/or a First Pass blood pool analysis. The transducers may be manually or automatically positioned proximate the candidate, for example, using a motorized or hydraulic drive mechanism. In this regard, the transducers may be automatically positioned using self-correcting positional transducer alignment. Further, operation of the transducers may be synchronized with operation of the ECG to provide ECG tracing signals concurrently with corresponding images of the heart captured by the transducers which may or may not provide synchronic-simultaneous imaging per transducer.

The transducers may acquire a plurality of images of the heart and the sensors may receive a plurality of ECG signals from the heart (Step 315). The ECG signals may include ECG tracing update signals and the plurality of images may include images that have been captured synchronously with the ECG tracing update signals, providing corresponding blood pool output data (Global and Regional). The acquired image data and ECG data may be processed to generate multi-screen format data for display and for monitoring and measuring changes in cardiac function of the heart (Step 320). On a basis of the processed data (Step 320), cardiac function values may be determined for a left ventricular ejection fraction, a right ventricular ejection fraction and a synchrony/asynchrony of the heart (Step 325). At substantially the same time, an analysis of a T-wave spectrum of ECG data may be performed to accurately calculate a T-wave spectrum in the heart to provide a T-wave value (Step 330). It is noted that the processes of Steps 325 and 330 may be performed in any order, including sequentially or substantially simultaneously.

For example, the cardiac function values may be determined by monitoring and measuring the activity of emitted particles from the radioactive tracer in the candidate's cardiac blood pool via a standard multi-gated acquisition (MUGA) analysis and/or a First Pass blood pool analysis. Further, the T-wave value may be determined by performing T-wave analysis using, e.g., a spectrum analyzer like the one provided by Cambridge Heart, Inc.™, which provides T-wave values in terms T-wave microvolts.

The determined cardiac function and T-wave values may be output for analysis by a user, such as, for example, but not limited to, a technician, a nurse, a physician, or the like (Step 335). Further, the results may be output as a display of a first Fourier Harmonic fit of the gated blood pool (First Pass or Equilibrium) time versus a radioactive curve. The results may also be output as a display of LV/RV Ventricular Volumes, Stroke Volumes, Cardiac Output, LV Segmental Wall Motion, or LV wall motion Speed and Distance from set a Region of Interest (ROI). The displayed phase angle may represent the timing of regional contraction of the candidate's heart. Moreover, the Regional and Global ventricular synchrony of the heart may be based on known phasic methods, which the skilled artisan will readily recognize and appreciate.

For example, from a fixed Region of Interest (ROI) of the heart, a speed and a distance of an endocardial wall motion of the Left Ventricle (LV) may be measured using, for example, reverse tissue Doppler. In this regard, a fixed region surrounding the LV may be drawn at Diastole and at Systole positions and then calculated for speed of contraction of the endocardial wall, as well as distance traveled. Resultantly, a determination may be made as to whether the Left Ventricle of the heart functions in synchronism with the Right Ventricle of the heart, and whether the Ejection Fraction of the LV and/or RV ventricles exceeds a predetermine threshold, such as, for example, thirty-five percent (35%) of volume.

A determination may be made whether to proceed with a further analysis of the heart (Step 340). The determination may be based on the determined cardiac function values, the T-wave values, or an instruction from the user. If a determination is made to proceed with a further analysis of the heart (Yes at Step 340), the candidate may be exposed to an increased stimulant, such as, for example, low level exercise (Step 345), and the process repeated (Step 315).

However, if a determination is made not to proceed with a further analysis of the heart (No at Step 340), a result of the screening process is output to the user and stored in a storage (not shown) for later use (Step 350). The result may include, for example, a left ventricular ejection fraction value, a right ventricular ejection fraction value, a synchrony/asynchrony value, a T-wave value, and the like, and possibly a recommendation for an implantable CSD, a particular type of CSD, as well as one or more CSD parameters, such as, e.g., a voltage (or amplitude) value, a frequency value, a phase value, and/or the like for the CSD.

Further, multi-gated equilibrium radionuclide angiography (MUGA) and Fourier phase analysis may be used, for example, in candidates with idiopathic dilated cardiomyopathy (DCM), where the QRS duration may be related to both interventricular and intraventricular asynchrony. Intraventricular asynchrony may be an independent predictor of a cardiac event (such as, e.g., cardiac death, worsening of HF and heart transplantation) in DCM candidates, where the prognosis may be found to be related to intraventricular rather than to interventricular asynchrony.

Phase imaging and the standard deviation of left ventricular phase angle may be a strong indicator of ventricular synchrony and prognosis in patients with severe congestive cardiomyopathy and heart failure. Moreover, it may aid in identifying heart failure candidates who might benefit most from the placement of cardiac stimulation devices (CSDs), such as, for example, biventricular pacemakers, and serve as a strong measure of the benefit of the CSD after device placement.

In the embodiment of the disclosure, a computer readable medium may be provided that includes a computer program, which when executed by, for example, the computer 270 (shown in FIG. 2), may cause the computer 270 to carry out each of the above Steps 310 through 350 shown in FIG. 3. Moreover, the computer program may have a segment of code for carrying out each of the Steps 310 through 350.

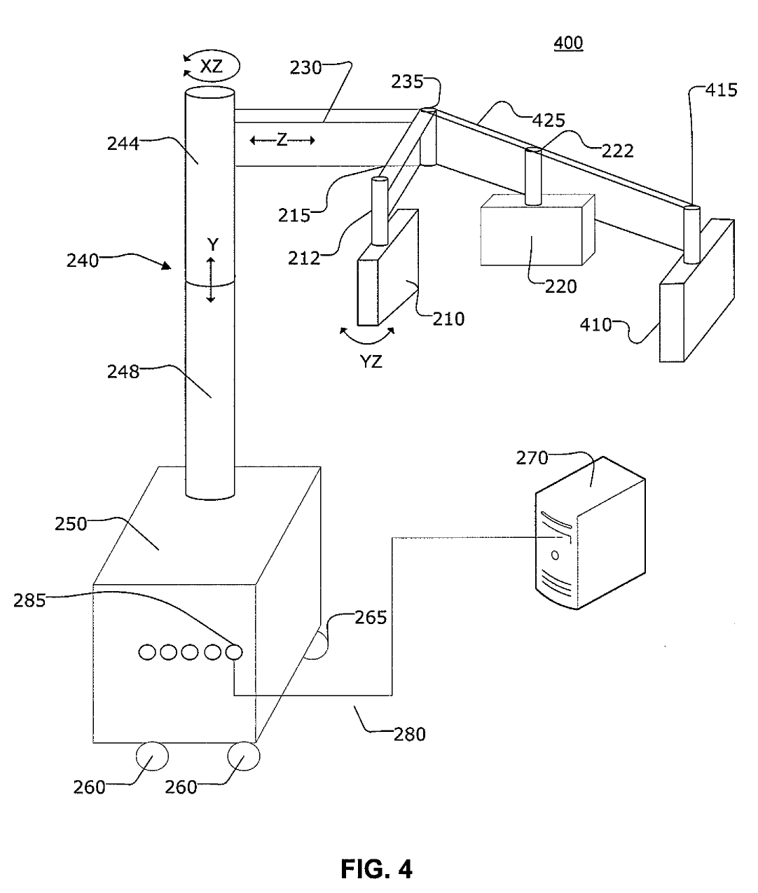

FIG. 4 shows an example of a candidate screening apparatus (CSA) 400 that includes three transducers 210, 220, 410. The third transducer 410 may be pivotally attached to a transducer support member 425 through a pivotal member 415. The pivotal member 415 may provide for manual or automatic rotational movement of the transducer 410 in the X-Z plane and/or manual or automatic pivotal movement in the Y-Z plane. The other elements in FIG. 4 may be the same as those disclosed in FIG. 2.

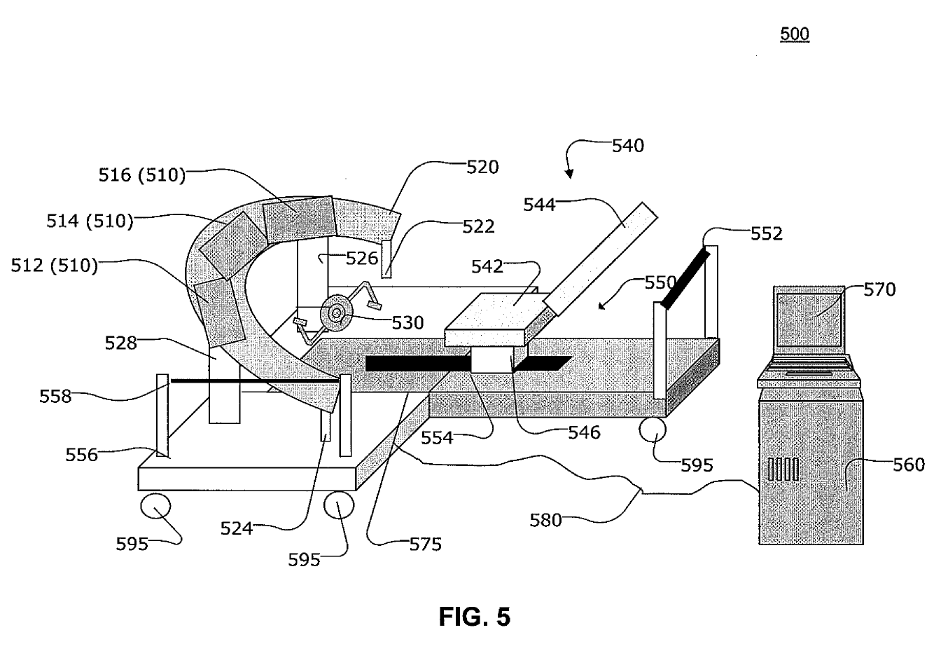

FIG. 5 shows an example of a candidate screening system (CSS) 500 for exposing the candidate to an increased stimulant, such as, for example, low level exercise, according to an embodiment of the disclosure. The CSS 500 may include a plurality (or an array) of transducers 510, a transducer array support 520, a physical resistance device 530, a candidate support member 540, a candidate platform 550, a processor housing 560 and a computer 570. The candidate platform 550 may include a candidate support platform 554 and a transducer array support platform 556, each of which may be separately moveable using, for example, handle bars 552, 558, or a motorized or a hydraulic drive mechanism (not shown).

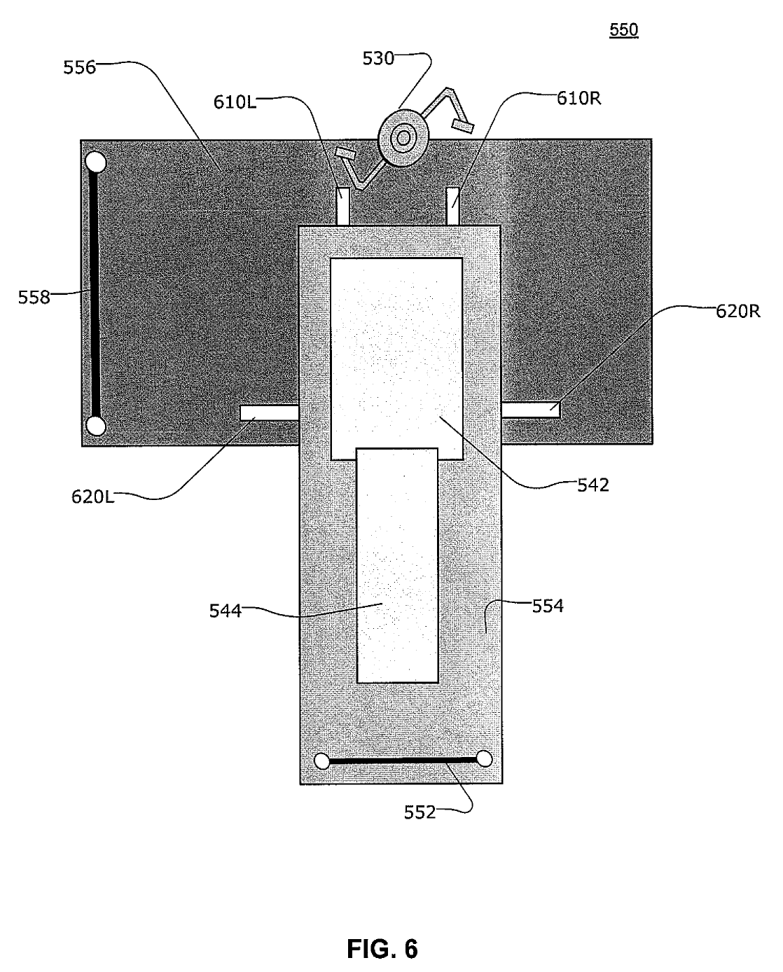

FIG. 6 shows an example of a preferred embodiment of the candidate platform 550, which may include the candidate support platform 554 and the transducer array support platform 556. The candidate support platform 554 and the transducer array support platform 556 may each include a plurality of coasters 595 (shown in FIG. 5) configured to move the candidate support platform 554 and/or the transducer array support platform 556 in any direction parallel to a floor surface (not shown). The coasters 595 may be moveable as result of a force applied to the handlebars 552, 558, or the coasters 595 may be driven by a motorized mechanism (not shown) or hydraulic mechanism (not shown).

As seen in FIG. 6, the candidate support platform 554 may be configured to slide into a recess (not shown) in the transducer array support platform 556. Alternatively or additionally, the candidate support platform 554 may be configured to slide over or under (or both over and under) the transducer array support platform 556. The candidate support platform 554 may be lockably engaged to the transducer array support platform 556 through a plurality of first engaging members 610, including a left engaging member 610L and a right engaging member 610R, and a plurality of second engaging members 620, including a left engaging member 620L and a right engaging member 620R. Each of the plurality of engaging members 610, 620 may be configured to include, but are not limited to, a pin, a magnetic coupling device, a bolt, a nut, a latch, a hinge, or the like.

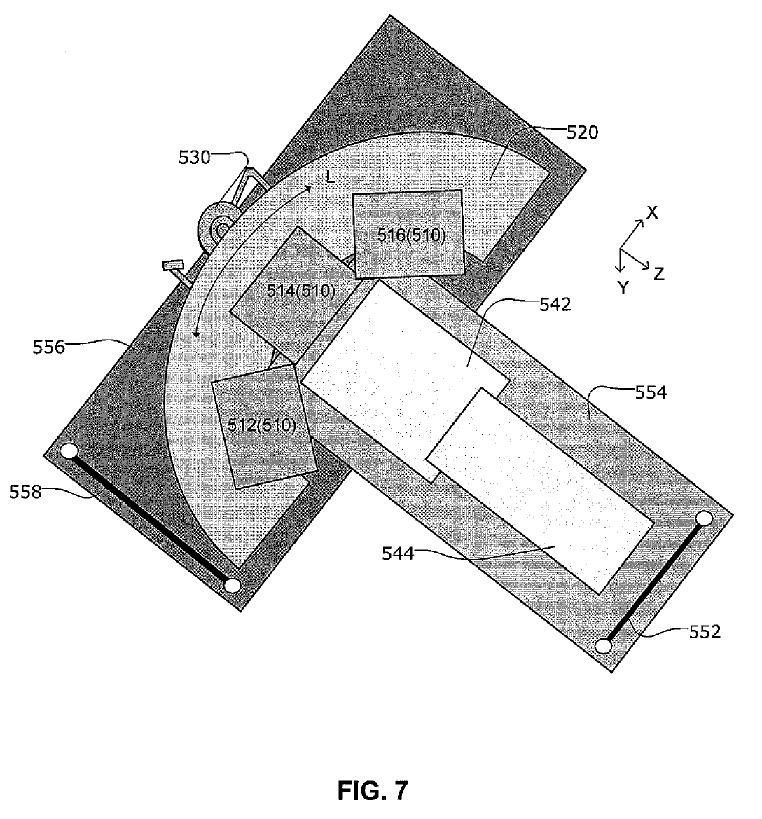

FIG. 7 shows a further example of a top view of the preferred embodiment of the candidate platform 550 together with the transducer array support 520, the physical resistance device 530 and the plurality of transducers 510.

Referring to FIG. 5 and FIG. 7, the candidate support member 540 may include a seat member 542 and/or a back member 544. The seat member 542 may be adjustably affixed to the candidate support platform 554 through an adjustable base 546, which may be configured to move in a vertical direction and/or a horizontal direction through a manual mechanism (not shown), a motorized mechanism (not shown), or a hydraulic mechanism (not shown). Thus, a candidate may be placed on the candidate support member 540 in a seated position, a supine position, a recumbent position, or a vertical position. The candidate support member 540 may be moved closer to or further from the transducer array support 520 through movement of the adjustable base 546. In this regard, the adjustable base 546 may be affixed to one or more slideable tracks (not shown) in the candidate support platform 554 that provide for manual or automated movement of the adjustable base 546. Additionally, the transducer array support 520 may be configured to move closer to, or further from the candidate support member 540.

The transducer array support 520 may include the plurality of transducers 510 adjustably mounted thereto, or integrated into the transducer array support 520. In either configuration, each of the plurality of transducers 510 (i.e., 512, 514, 516) may be moveable along the length of the transducer array support 520 in a direction L (shown in FIG. 7). Moreover, each of the plurality of transducers 510 may be moveable so as to move in any direction in the three-dimensional world coordinate system (x, y, z). The transducers 510 may be configured to move individually or as a group, each transducer 512, 514, 516 being moveable through a manual drive mechanism (not shown), a motorized drive mechanism (not shown), or a hydraulic drive mechanism (not shown).

Furthermore, the transducer array support 520 may be adjustably affixed to the transducer array support platform 556 through a plurality of broad-range posts 526, 528 and a plurality of narrow-range posts 522, 524. The broad-range posts 526, 528 may include any one or more of a manual drive mechanism (not shown), a motorized drive mechanism (not shown) or a hydraulic drive mechanism (not shown), which may be configured to provide precise large scale movement of the broad-range posts 526, 528. Each of the broad-range posts 526, 528 may be pivotally attached to the transducer array support 556 at one end and the transducer array support 520 at the other end. The broad-range posts 526, 528 may provide large scale movement of the transducer array support 520 through, for example, contraction or expansion of the broad-range posts 526, 528.

Similarly, the narrow-range posts 522, 524 may include any one or more of a manual drive mechanism (not shown), a motorized drive mechanism (not shown), or a hydraulic drive mechanism (not shown), which may be configured to provide precise small scale movement of the narrow-range posts 522, 524. Each of the narrow-range posts 522, 524 may be pivotally attached to the transducer array support 556 at one end and the transducer array support 520 at the other end. The narrow-range posts 522, 524 may provide small scale movement of the transducer array support 520. Alternatively (or additionally), the narrow-range posts 522, 524 may include hinged brackets, or the like.

Through selective control of the broad-range posts 526, 528 and/or the narrow-range posts 522, 524, the transducer array support 520 may be precisely positioned in any one of a wide range of discrete positions with respect to a candidate placed on the candidate support member 540. Each of the broad-range posts 526, 528 and the narrow-range posts 522, 524 may be moved individually or simultaneously as a group. Accordingly, the transducer array support 520 may be precisely located with respect to the candidate so as to position the transducers 512, 514, 516 proximate the chest of the candidate, and the transducers 512, 514, 516 may be located in respective positions with respect to the candidate's chest to provide optimized functionality.

The physical resistance device 530 may include a pair of foot pedals as seen in FIG. 5. Alternatively, the physical resistance device 530 may include a pair of ski-like members slideably placed in a pair of tracks (not shown), or any other device capable of manipulation by a candidate to cause physical exertion by the candidate to elevate the candidate's heart rate, without limitation. In this regard, the physical resistance device 530 is not limited to manipulation by a foot of a candidate, but may, instead (or in addition) include a mechanism that may be manipulated by a hand(s) of the candidate. The negative force exerted by the physical resistance device 530 may be adjustable depending on an age, a physical condition, a gender, a weight, a body mass, medical history, family medical history, or the like, of the candidate.

The candidate support platform 554 and transducer array support platform 556 may each include a plurality of electric contacts (not shown) and an electrical input/output (IO) interface (not shown). For example, each of the candidate support platform 554 and the transducer array support platform 556 may include a plurality of contact electrodes (not shown) and electrical contacts (not shown) on a contact surface 575 where a surface of the candidate support platform 554 slideably engages (or contacts) a surface of the transducer array support platform 556. Further, each of the candidate support platform 554 and the transducer array support platform 556 may include one or more of a plurality of male-female electrical coupling pairs. Furthermore, each of the candidate support platform 554 and the transducer array support platform 556 may include a wireless communication device (not shown), a dedicated processor device (not shown) and a power supply (not shown) for wirelessly communicating with each other, as well as other component devices such as, e.g., but not limited to, the computer 570 and/or the processor housing 560.

The candidate platform 550 may be coupled to the processor housing 560 and/or the computer 570 through a communication medium 580, such as, e.g., a wired communication medium, a wireless communication medium, or a combination of a wired and wireless communication medium. ECG tracing updates with corresponding Blood Pool output data (Global and Regional) may be provided from the candidate platform 550 to the processor housing 560 and/or the computer 570 through the communication medium 580. The ECG tracing updates with corresponding Blood Pool output data may be displayed in a multi-screen format on a display of, for example, the computer 570, for wall motion and phasic analysis-comparative updates per sequence of optimization.

The processor housing 560 may include, for example, a plurality of ports (not shown), an IO interface (not shown), a computer (not shown), a storage (not shown), and the like. The computer may be similar to, or the same as the computer 270 (shown in FIG. 2), including any machine, device, circuit, component, or module, or any system of machines, devices, circuits, components, modules, or the like, which are capable of manipulating data according to one or more instructions, such as, for example, without limitation, a processor, a microprocessor, a central processing unit, a general purpose computer, a personal computer, a laptop computer, a palmtop computer, a notebook computer, a desktop computer, a workstation computer, a server, or the like, or an array of processors, microprocessors, central processing units, general purpose computers, personal computers, laptop computers, palmtop computers, notebook computers, desktop computers, workstation computers, servers, or the like.

The computer 570 may be similar to, or the same as the computer 270 (shown in FIG. 2). Similarly, the computer 570 may include any machine, device, circuit, component, or module, or any system of machines, devices, circuits, components, modules, or the like, which are capable of manipulating data according to one or more instructions, such as, for example, without limitation, a processor, a microprocessor, a central processing unit, a general purpose computer, a personal computer, a laptop computer, a palmtop computer, a notebook computer, a desktop computer, a workstation computer, a server, or the like, or an array of processors, microprocessors, central processing units, general purpose computers, personal computers, laptop computers, palmtop computers, notebook computers, desktop computers, workstation computers, servers, or the like.

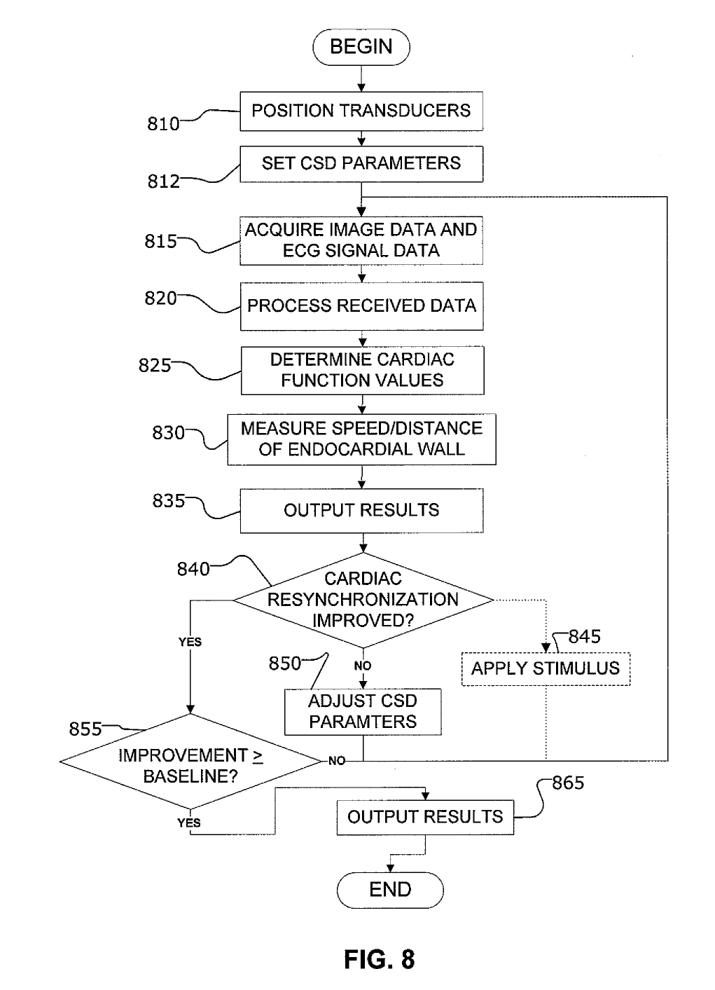

FIG. 8 shows an example of a process for evaluating heart function in candidates with implanted CSDs and for optimizing the implanted CSDs, according to an embodiment of the disclosure.

Referring to FIG. 8, after a candidate with an implanted CSD has been injected with one of several radionuclide tracers, one or more transducers may be positioned proximate the candidate and a plurality of sensors may be connected to the candidate (Step 810). The sensors, which may include high-resolution electrodes, may be coupled to an electrocardiogram (ECG). The transducers may be positioned in close proximity to the chest and heart of the candidate from several different projections, thereby making it possible to acquire data for a standard multi-gated acquisition (MUGA) analysis and/or a First Pass blood pool analysis. The transducers may be manually or automatically positioned proximate the candidate, for example, using a manual, a motorized or a hydraulic drive mechanism (not shown). In this regard, the transducers may be automatically positioned using self-correcting positional transducer alignment, for example, under control of the processor housing 560, the computer 570, or a processor (not shown) provided in some other location of the CSS 500. Further, operation of the transducers may be synchronized with operation of the ECG to provide ECG tracing signals concurrently with corresponding images of the heart captured by the transducers.

An operation mode, including CSD parameters, of the implanted CSD device may be set to a baseline, such as, e.g., a standard PR interval, by setting, for example, a voltage value, a frequency value, a phase value, or the like, of the CSD (Step 812). Alternatively (or additionally) the CSD parameters may be acquired from the candidate and set as the CSD parameters (Step 812), or the CSD parameters may be acquired from a database (not shown) having previously stored CSD parameters for the particular candidate and set as the CSD parameters (Step 812).

The transducers may acquire image data by capturing a plurality of images of the heart and the sensors may provide ECG data by receiving a plurality of ECG signals from the heart (Step 815). The ECG signals may include ECG tracing update signals and the plurality of images may include images that have been captured synchronously with the ECG tracing update signals, providing corresponding Blood Pool output data (Global and Regional). The acquired image data and ECG data may be processed to generate multi-screen format data for display and for monitoring and measuring changes in cardiac function of the heart (Step 820). On a basis of the processed data (Step 820), cardiac function values may be determined for a left ventricular ejection fraction, a right ventricular ejection fraction and a synchrony/asynchrony of the heart (Step 825). Further, on the basis of the processed data, a fixed region of interest of the candidate's heart may be measured in terms of a speed of motion and a distance of motion of an endocardial wall using, e.g., but not limited to, reverse tissue Doppler (Step 830). For example, the velocity of contraction to compare lag time between Septal and LV posterior wall contraction may be analyzed. In this regard, a fixed region surrounding a Left Ventricle may be generated at Diastole position and at Systole position and then a speed of contraction and a distance traveled of the endocardial wall may be measured. Resultantly, a determination may be made as to whether the Left Ventricle of the heart functions in synchronism with the Right Ventricle of the heart, and whether the Ejection Fraction of the LV and/or RV ventricles exceeds a predetermine threshold, such as, for example, but not limited to, thirty-five percent (35%) of volume.

For example, the cardiac function values may be determined (Step 825) by monitoring and measuring the activity of emitted particles from the radioactive tracer in the candidate's cardiac blood pool via a standard multi-gated acquisition (MUGA) analysis and/or a First Pass blood pool analysis and monitoring and measuring the associated ECG data from the candidate's heart. The determined cardiac function values may be output for analysis by a user, such as, for example, but not limited to, a technician, a nurse, a physician, or the like (Step 835). Further, the results may be output as a display of a first Fourier Harmonic fit of the gated blood pool (First Pass or Equilibrium) time versus a radioactive curve. The results may also be output as, for example, but not limited to, a display of LV/RV Ventricular Volumes, Stroke Volumes, Cardiac Output, LV Segmental Wall Motion, or LV wall motion Speed and Distance from a set Region of Interest (ROI). The displayed phase angle may represent the timing of regional contraction of the candidate's heart. Moreover, the Regional and Global ventricular synchrony of the heart may be based on phasic methods.

A determination may be made whether the Cardiac Resynchronization has improved (Step 840). The determination may be based on, for example, but is not limited to, the determined cardiac function values, the speed and/or distance of the endocardial wall motion, or an instruction from the user. If a determination is made that the Cardiac Resynchronization has improved (Yes at Step 840), then a determination may be made as to whether the improvement is greater than or equal to a baseline (Step 855), otherwise one or more parameters of the CSD device are adjusted (Step 850) and the process is repeated (Step 815). If a determination is made that the improvement of the Cardiac Resynchronization is greater than or equal to the baseline (Yes at Step 855), then the results are output (Step 865) and the process may end, otherwise the process may repeat (Step 815).

Optionally, a stimulus, such as, e.g., but not limited to, exercise may be applied to the candidate (Step 845) and the process repeated (Step 815). The stimulus may be applied automatically based on a result of the determination of whether cardiac resynchronization has improved (Step 840), an instruction from the user, a predetermined schedule, or the like. Additionally, the stimulus may be applied before or after the one or more parameters of the CSD device are adjusted (Step 850), before or after the determination is made whether the improvement was greater than or equal to the baseline (Step 855), or before or after the results are output (Step 865). The stimulus may include, for example, but is not limited to, exposing the candidate to incrementally increasing stimulation, such as, e.g., low level exercise during, or before repeating, the process shown in FIG. 8.

In an embodiment of the disclosure, a computer readable medium may be provided that includes a computer program, which when executed by, for example, the computer 270 (shown in FIG. 2) or the computer 570 (shown in FIG. 5), may cause the computer to carry out each of the above Steps 810 through 865 shown in FIG. 8. Moreover, the computer program may include a segment of code for carrying out each of the Steps 810 through 865.

The process for evaluating heart function in candidates with implanted CSDs and for optimizing the implanted CSDs (shown in FIG. 8) may be carried out to ensure adequate CSD device function in a CRT candidate with persistent or worsening symptoms. This may lead to evaluation of atrioventricular (AV) and interventricular (VV) delay. Restoration of optimal AV timing may improve systolic performance by optimizing left ventricular preload.

Selecting candidates with mechanical LV asynchrony prior to implanting a CSD device may improve the LV function deteriorated by asynchrony of using the CSD device. Once selected, this aspect of the candidate's condition may be investigated using the above process (FIG. 8), including but not limited to, for example, assessing the candidate's LV functional parameters.

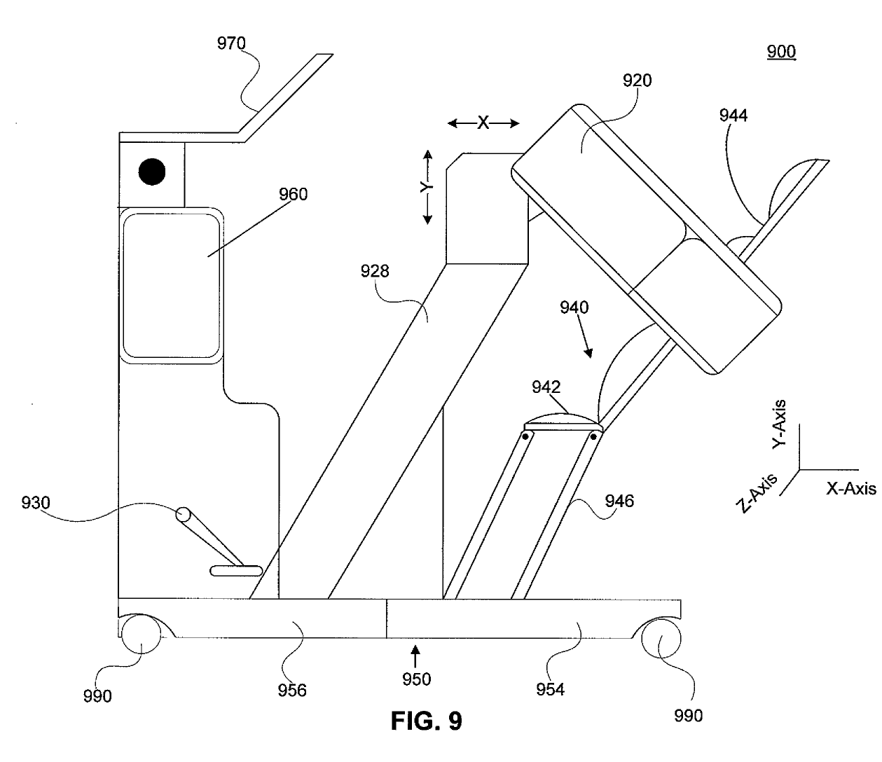

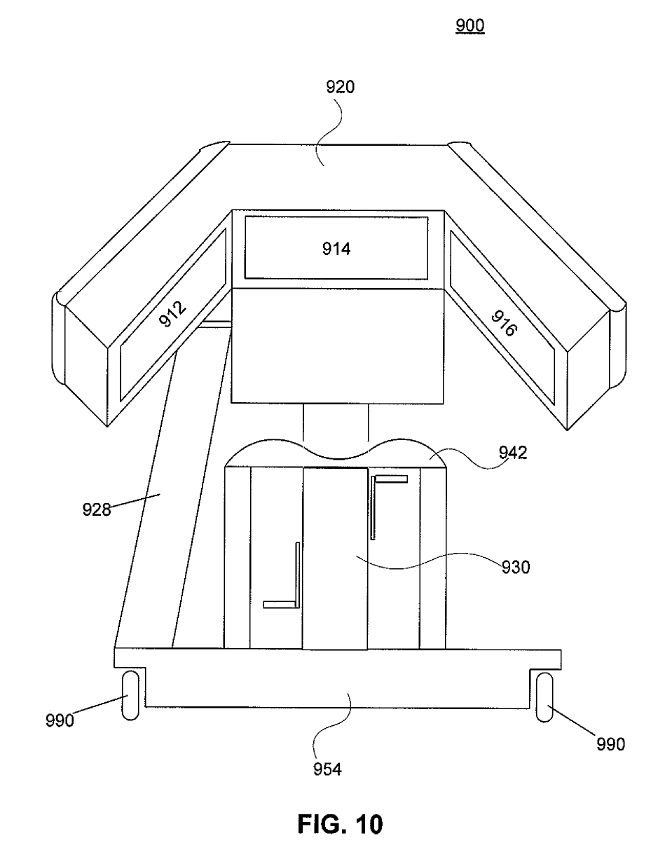

FIG. 9 shows another example of a candidate screening/optimization system (CSS) 900 that may be configured as a single mobile unit from multiple units. The CSS 900 may include a plurality (or an array) of transducers 912, 914, 916 (shown in FIG. 10), a transducer array support 920, a physical resistance device 930, a candidate support member 940, a candidate platform 950, a processor housing 960 and a computer 970. The candidate platform 950 may be configured as a single platform, or it may include a candidate support platform 954 and a transducer array support platform 956, each of which may be separately moveable using, for example, but not limited to, a manual, a motorized, or a hydraulic drive mechanism (not shown).

The candidate support member 940 may include a seat member 942 and/or a back member 944. The seat member 942 may be adjustably affixed to the candidate platform 950 through, for example, but not limited to, a plurality of adjustable seat support members 946, which may be configured to move in a vertical direction and/or a horizontal direction through a manual drive mechanism (not shown), a motorized drive mechanism (not shown), or a hydraulic drive mechanism (not shown). Thus, a candidate may be placed on the candidate support member 940 in a seated position, a supine position, a recumbent position, or a vertical position. The candidate support member 940 may be moved closer to or further from the transducer array support 920 through movement of the adjustable seat support members 946. In this regard, the adjustable seat support members 946 may be affixed to one or more slideable tracks (not shown) in the candidate platform 950 that provide for manual or automated movement of the adjustable seat support members 946. Additionally, the transducer array support 920 may be configured to move closer to, or further from the candidate support member 940 in a direction X, rotate pivotally around the candidate support member 940 in the Y-Z plane, or pivot up or down with respect to the candidate support member 940 in the X-Y plane.

The transducer array support 920 may include the plurality of transducers 912, 914, 916 mounted thereto, or integrated into the transducer array support 920. In either configuration, each of the plurality of transducers 912, 914, 916 may be moveable individually or as a group, or the transducers 912, 914, 916 may be fixed. The transducers 912, 914, 916 may be moveable through a manual drive mechanism (not shown), a motorized drive mechanism (not shown), or a hydraulic drive mechanism (not shown). Additionally, the image signal captured by the transducers 912, 914, 916 may be manipulated electronically to change the area of image pickup, a zoom-in or zoom-out of the area of image-pickup, or the like.

Furthermore, the transducer array support 920 may be adjustably affixed to the candidate platform 950 through a single broad-range post 928, or a plurality of broad-range posts (as shown, e.g., in FIG. 5). The broad-range post 928 may include any one or more of a manual drive mechanism (not shown), a motorized drive mechanism (not shown) or a hydraulic drive mechanism (snot shown), which may be configured to provide precise movement of the broad-range post 928. The broad-range post 928 may be pivotally attached to the transducer array support 920 at one end and the candidate platform 950 through a manual drive mechanism (not shown), a motorized mechanism (not shown) or a hydraulic mechanism (not shown) at either, or both ends. Alternatively, the broad-range post 928 may be fixedly attached to the transducer array support 920 and the candidate platform 950. The broad-range post 928 may provide precise movement of the transducer array support 920 through, for example, contraction or expansion of the broad-range post 928.

The processor housing 960 and the computer 970 may be similar to or the same as the processor housing 560 and the computer of 570 of FIG. 5, respectively.

FIG. 10 shows a rear view of the candidate screening/optimization system (CSS) of FIG. 9.

It is noted that each of the components disclosed herein as moveable may include a manual drive mechanism, a motorized drive mechanism or a hydraulic drive mechanism, or any proper combination thereof without limitation. Each of the manual drive mechanisms disclosed herein may include, but are in no way limited to, one or more gears, a belt, a chain, a lever, a track, or any other mechanism capable of precise movement of an object through manual manipulation. Further, each of the motorized drive mechanisms disclosed herein may include, but are in no way limited to, one or more gears, a motor, a controller, a belt, a chain, a track, and/or any other mechanism capable of precise motorized movement of an object through manual or automatic control of one or more motors. Furthermore, each of the hydraulic mechanisms disclosed herein may include, but are in no way limited to, one or more gears, a piston, a cylinder, a compressible liquid or gas substance, a pump, a valve, or any other mechanism capable of precise movement of an object through manual or automatic control of a characteristic of a liquid or gas substance, such as, but not limited to, pressure, temperature, or the like.

While the disclosure has been described in terms of example embodiments, those skilled in the art will recognize that the disclosure can be practiced with switchable modifications in the spirit and scope of the appended claims. These examples given above are merely illustrative and are not meant to be an exhaustive list of all possible designs, embodiments, applications or modifications of the disclosure.

What is claimed is:

1. An apparatus for optimizing an implanted device in a candidate, the apparatus comprising:
a first sensor configured to sense a tracing signal;
a transducer configured to capture an image of a region of interest, where the image is captured in synchronism with the tracing signal; and
a determiner configured to determine a cardiac functional value based on the image and the tracing signal,
wherein a parameter of the implanted device is adjusted based on the cardiac functional value.

2. The apparatus of claim 1, further comprising:
a second transducer configured to capture a second image of the region of interest, where the second image is captured in synchronism with the tracing signal.

3. The apparatus of claim 2, further comprising:
a third transducer configured to capture a third image of the region of interest, where the third image is captured in synchronism with the tracing signal.

4. The apparatus of claim 2, further comprising:
a support member configured to moveably support the transducer and the second transducer.

5. The apparatus of claim 4, wherein the transducer and the second transducer are moveable based on the region of interest.

6. The apparatus of claim 1, further comprising:
a mobile candidate support; and
a mobile transducer array support,
wherein the mobile candidate support and the mobile transducer array support are lockably engageable.

7. The apparatus of claim 6, wherein the mobile candidate support platform comprises an adjustable candidate support member.

8. The apparatus of claim 7, wherein the adjustable candidate support member is configurable in a seat position, a supine position, a recumbent position, or a vertical position.

9. The apparatus of claim 1, further comprising:
a physical resistance device configured to provide a resistive force.

10. The apparatus of claim 9, wherein the physical resistance device comprises at least one foot pedal.

11. The apparatus of claim 4, further comprising:
a broad-range post configured to provide substantially precise movement of the support member.

12. The apparatus of claim 11, further comprising:
a narrow-range post configured to provide substantially precise movement of the support member.

13. The apparatus of claim 11, wherein the narrow-range post comprises a hinged bracket.

14. The apparatus of claim 11, wherein the broad-range post comprises a motorized post.

15. The apparatus of claim 6, wherein the mobile candidate support and the mobile transducer array support are lockable by at least one locking pin.

16. The apparatus of claim 1, wherein the tracing signal comprises an electrocardiogram tracing update signal.

17. A method for evaluating an implanted device in a candidate, the method comprising:
sensing a tracing signal;
capturing an image of a region of interest in synchronism with the tracing signal;
determining a cardiac functional value based on the tracing signal and the image; and
adjusting a parameter of the implanted device based on the cardiac functional value.

18. The method of claim 17, further comprising:
capturing a second image of the region of interest in synchronism with the tracing signal.

19. The method of claim 18, further comprising:
capturing a third image of the region of interest in synchronism with the tracing signal.

20. The method of claim 17, further comprising:
moveably supporting a first transducer and a second transducer to capture said image and said second image.

21. The method of claim 20, further comprising:
moving the first transducer and the second transducer based on the region of interest.

22. The method of claim 20, further comprising:
moving a candidate support platform to position a candidate proximate the first transducer and the second transducer.

23. The method of claim 17, further comprising:
providing a resistive force to a candidate to cause an elevated heart rate.

24. The method of claim 20, wherein said moveably supporting comprises:
providing substantially precise large scale movement of a support member; and
providing substantially precise small scale movement of the support member.

25. The method of claim 24, wherein the small scale movement comprises moving a hinged bracket.

26. The method of claim 24, wherein the large scale movement comprises controlling a motorized post.

27. The method of claim 17, further comprising:
determining whether cardiac synchronization is improved based on the cardiac functional value.

28. A computer readable medium comprising a program for evaluating an implanted device in a candidate, the medium comprising:
a sensing code segment that, when executed by a computer, causes sensing a tracing signal;
an image capturing code segment that, when executed by the computer, causes capturing an image of a region of interest in synchronism with the tracing signal;
a cardiac functional value determining code segment that, when executed by the computer, causes determining a cardiac functional value based on the tracing signal and the image;
a parameter adjusting code segment that, when executed by the computer, causes adjusting a parameter of the implanted device based on the cardiac functional value; and
an improvement determining code segment that, when executed by the computer, causes determining whether a cardiac synchronization improvement is greater than or equal to a baseline based on the cardiac functional value.
